(19) United States
(12) Patent Application Publication
(10) Pub. No.: US 2020/0170698 A1
(43) Pub. Date: Jun. 4, 2020

(54) MEDICAL TORQUE HAND TOOL

(71) Applicant: METAL INDUSTRIES RESEARCH & DEVELOPMENT CENTRE, KAOHSIUNG CITY (TW)

(72) Inventors: CHE-AN PAI, KAOHSIUNG CITY (TW); MING FENG WU, KAOHSIUNG CITY (TW); HAO-YUAN TANG, TAIPEI CITY (TW); HSIANG-HO CHEN, NEW TAIPEI CITY (TW); CHING-AN LIN, KAOHSIUNG CITY (TW)

(21) Appl. No.: 16/699,217

(22) Filed: Nov. 29, 2019

(30) Foreign Application Priority Data
Nov. 30, 2018 (TW) 107143138

(51) Int. Cl. A61B 17/88

(52) U.S. Cl. CPC A61B 17/8875; A61B 2090/031

(57) ABSTRACT

The present invention provides a medical torque hand tool, which comprises a holding part, a torque adjusting device, a torque damping device, and a surgery tool. The holding part is disposed in a hollow member. The torque damping device is disposed in said holding part. The torque adjusting device passes through said holding part. The torque adjusting device and the torque damping device control the elastic deformation of an elastic member for controlling the torque of screwing an object.

### FIELD OF THE INVENTION

The present invention relates generally to a hand tool, and particularly to a medical torque hand tool.

### BACKGROUND OF THE INVENTION

The human skeleton is soft, elastic, and shapeable when just formed. As people age, due to the degeneration of neural system or the influence of external force (for example, traffic accidents), fractures might occur and subsequent therapies are required. In modern surgical technologies, orthopedists operate various mechanical tools to enter human bodies for performing many surgeries such as repairing bone fractures or implanting artificial joints.

In orthopedic surgeries, fixation components such as absorbable bone screws, bone plates, intramedullary nails, suture rivets are usually adopted. The hand tools used in the surgeries according to the prior art are unable to limit the torque value of the tools. Screwing in surgeries all relies on operators' hand feel.

Unfortunately, owing to the difference in force control, the screwing force will differ, resulting in loosing of bone screws, bone plates, suture rivets in human body and, in turn, leading to reoperations. For elderly patients, reoperations are long rehabilitation processes, and lead to economic pressure and related problems for patients' families.

In addition, the tools might contact blood in operations. To avoid infection caused by hand tools contaminated by bodily fluid, high-temperature and high-pressure sterilization is required. The cleaning and storage methods for hand tools should be considered. They should be sterilized in high temperatures and stored individually.

To improve the above problems, the present invention provides a hand tool for surgeries. By using the medical hand tool according to the present invention, the screwing torque can be controlled for screwing suture rivets in surgeries.

### SUMMARY

An objective of the present invention is to provide a medical torque hand tool. By fine tuning a torque adjusting device to set the torque value of the hand tool, the bone screws and bone plates with identical conditions in a surgery can be screwed rapidly.

Another objective of the present invention is to provide a medical torque hand tool, in which a through member goes through the hand tool for screwing suture rivets using a fixed torque value in a surgery.

In order to achieve the above objectives, the present invention discloses a medical torque hand tool, which comprises a torque adjusting device, a holding part, and a torque damping device. The torque adjusting device includes an adjusting rod and an elastic member. The adjusting rod includes a first hole and is against the elastic member. The holding part includes a body and a blocking member. The body includes an accommodating space with a first opening on one end and a second opening on the other. The blocking member include a bearing hole and is connected to the second opening. The adjusting rod inserts the first opening. The elastic member is disposed in the accommodating space. The torque damping device is disposed in the accommodating space. The adjusting rod compresses and distorts the elastic member to press against the torque damping device. The torque damping device includes a penetrating part communicating the first hole. When a user exerts an torque force on the holding part to screw an object, due to the reaction force, the torque damping device produces a torque. When the torque is greater than the torque force, the torque damping device drives a tool to rotate along the direction of the torque force for screwing the object. When the torque force is greater than the torque, the torque damping device turns idly. Hence, the tool will not rotate.

According to an embodiment of the present invention, the first opening further includes an inner thread on the inner side, and the adjusting post further includes an outer thread on the outer side. The outer thread of the adjusting post matches the inner thread of the first opening for adjusting the spring force of the elastic member pressing the torque damping device.

According to an embodiment of the present invention, the torque damping device includes a sliding block. The sliding block includes a block hole, an undulate structure surrounding the block hole, and a driving shaft body. The undulate structure includes a plurality of slopes and a plurality of recesses arranged at intervals. The driving shaft body includes a spin shaft and one or more protrudent pillar. The penetrating part is disposed at the spin shaft. An assembly hole is disposed at one end of the spin shaft. The same end of the spin shaft is disposed in the block hole and the bearing hole. The one or more protrudent pillar is disposed on one side of the spin shaft. The one or more protrudent pillar is wedged in one or the plurality of recesses.

According to an embodiment of the present invention, the sliding block and the elastic member clip the protrudent pillars.

According to an embodiment of the present invention, the sliding block and the blocking member clip the protrudent pillars.

According to an embodiment of the present invention, a surgery tool passing through the hole of the torque damping device is also disclosed.

According to an embodiment of the present invention, the other end of the spin shaft includes a counter bore opposing to the first hole. The assembly hole and the counter bore form the penetrating part. The surgery tool includes a second hole. A through member passes through the first hole, the penetrating part, and the second hole.

According to an embodiment of the present invention, a hollow member is further disclosed. The hollow member envelops one side of the holding part.

### BRIEF DESCRIPTION OF THE DRAWINGS

FIG. 1 shows a stereoscopic diagram according to an embodiment of the present invention;

FIG. 2 shows an exploded view according to an embodiment of the present invention;

FIG. 3 shows a cross-sectional view according to an embodiment of the present invention;

FIG. 4 shows a cross-sectional view according to an embodiment of the present invention;

FIG. 5A shows a schematic diagram of the operation according to an embodiment of the present invention;

FIG. 5B shows a schematic diagram of the operation according to an embodiment of the present invention;

FIG. 6 shows a cross-sectional view according to an embodiment of the present invention; and

FIG. 7 shows a cross-sectional view according to an embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures. The concepts of the present invention can be embodied by various forms. Those embodiments are not used to limit the scope and range of the present invention.

The present invention improves the drawbacks of complicated operations and maintenance in the hand tools with adjustable torque according to the prior art. In addition, in the structure according to the prior art, lubricant is required, which is disadvantageous to surgeries. For operation usage, germ-free condition is mandatory and the tool can sustain high-temperature and high-pressure sterilization. The hand tools according to the prior art generally adopt normal mechanical operations, leading to incompliance with the standard for medical application and inconvenience in surgeries.

By research and development, the present invention provides a hand tool with adjustable torque, which comprises a torque adjusting device, a holding part, and a torque damping device. The holding part contains the torque adjusting device and the torque damping device. The torque adjusting device includes an elastic member, which is a device for adjusting torque. The torque damping device includes a sliding block and a driving shaft body. By using the elastic member to press the sliding block and the driving shaft body, the protrudent pillars of the driving shaft body will be fixed to a plurality of recesses of the sliding block. Then, the holding part is turned for driving the driving shaft body to screw an object. If the torque force is greater than the torque produced by the elastic member, the protrudent pillars of the driving shaft body will slide off the plurality of recesses to make the hand tool turn idly, and thus achieving the effect of fixing the torque.

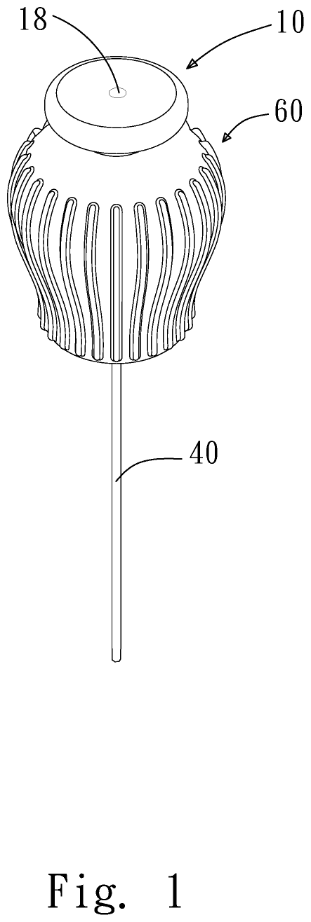

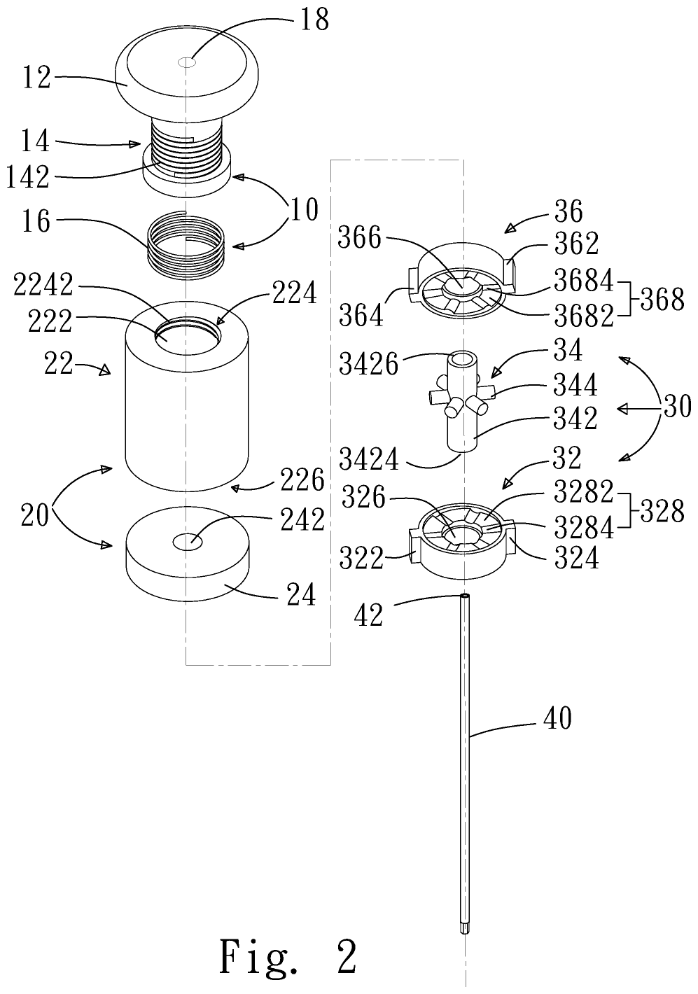

First, please refer to FIG. 1 and FIG. 2, which show a stereoscopic diagram and an exploded view, respectively, according to a preferred embodiment of the present invention. As shown in FIG. 1 and FIG. 2, the present invention provides a hand tool with adjustable torque, which comprises a torque adjusting device 10, a holding part 20, a torque damping device 30, a surgery tool 40, and a hollow member 60.

The torque adjusting device 10 includes a handheld part 12, an adjusting post 14, and an elastic member 16. The adjusting post 14 is against the elastic member 16. The adjusting post 14 further includes an outer thread 142. A first hole 18 passes through the handheld part 12 and the adjusting post 14. The holding part 20 includes a body 22 and a blocking member 24. The body includes an accommodating space 222, a first opening 224, and a second opening 226. The first opening 224 further includes an inner thread 2242. The blocking member includes a bearing hole 242. The elastic member 16 and the torque damping device 30 are disposed in the accommodating space 222. One end of the torque damping device 30 is against the elastic member 16 while the other end against the blocking member 24. By matching the outer thread 142 and the inner thread 2242 in the first opening 224, the adjusting post 14 can adjust the spring force of the elastic member 16 against the torque damping device 30. The surgery tool 40 includes a second hole 42 and passes through the torque damping device 30. The second hole 42 communicates the first hole 18. The surgery tool 40 can be a replaceable component. The post can be replaced according to surgery requirements.

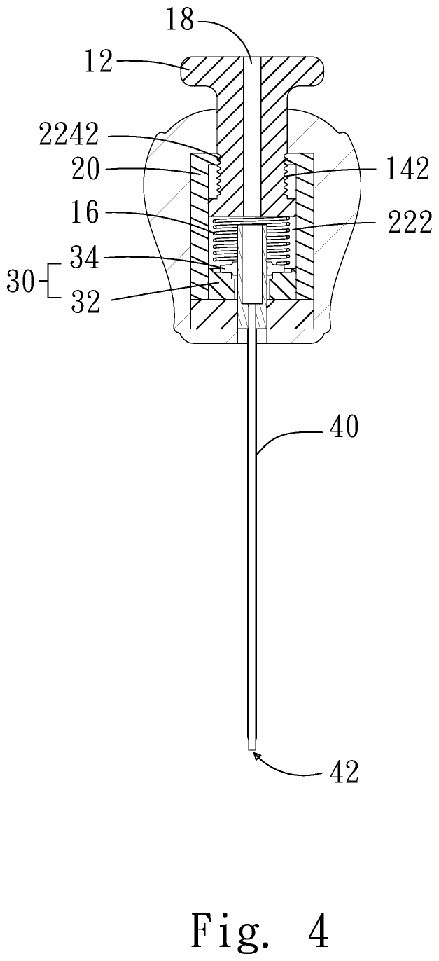

Please refer to FIG. 2 and FIG. 4. FIG. 4 shows a cross-sectional view according to an embodiment of the present invention. The torque damping device 30 includes a first sliding block 32 and a driving shaft body 34. The first sliding block 32 includes a first block hole 326 and a first undulate structure 328 surrounding the first block hole 326. The first sliding block 32 includes a first external ear part 322 and a second external ear part 324 on both sides for wedging into the body 22 such that the first sliding block 32 cannot rotate with respect to the body 22. The first undulate structure 328 includes a plurality of slopes 3282 and a plurality of recesses 3284 arranged at intervals. The driving shaft body 34 includes a spin shaft 342 and one or more protrudent pillar 344. The spin shaft 342 includes a penetrating part communicating the first hole. One end of the spin shaft 342 (the penetrating part) includes an assembly hole 3424. The other end of the spin shaft 342 (the penetrating part) includes a counter bore 3426. The assembly hole 3424 penetrates to the counter bore 3426 at the other end to form the penetrating part. The protrudent pillars 344 are disposed on one side of the spin shaft 342. The first sliding block 32 and the elastic member 16 clip the protrudent pillars 344. The driving shaft body 34 passes though the bearing hole 242 and the first block hole 326. The one or more protrudent pillars 344 are wedged into one of the plurality of recesses 3284.

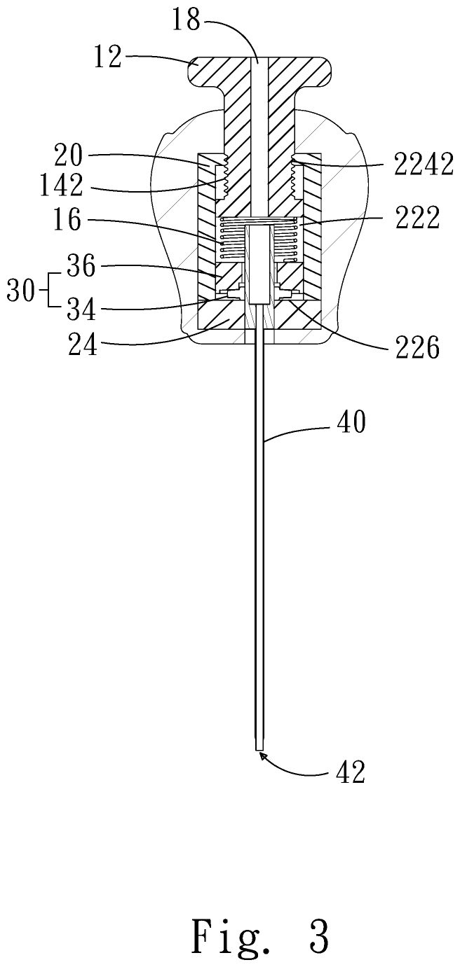

Next, please refer to FIG. 2 and FIG. 3. FIG. 3 shows a cross-sectional view according to an embodiment of the present invention. The torque damping device 30 includes a second sliding block 36 and the driving shaft body 34. The second sliding block 36 includes a second block hole 366 and a second undulate structure 368 surrounding the second block hole 366. The second sliding block 36 includes a third external ear part 362 and a fourth external ear part 364 on both sides for wedging into the body 22 such that the second sliding block 36 cannot rotate with respect to the body 22. The second undulate structure 368 includes a plurality of slopes 3682 and a plurality of recesses 3684. The driving shaft body 34 includes a spin shaft 342 and one or more protrudent pillar 344. One end of the spin shaft 342 includes an assembly hole 3424. The other end of the spin shaft 342 includes a counter bore 3426. The assembly hole 3424 penetrates to the counter bore 3426 at the other end. The protrudent pillars 344 are disposed on one side of the spin shaft 342. The second sliding block 36 and the blocking member 24 clip the protrudent pillars 344. The driving shaft body 34 passes though the bearing hole 242 and the second block hole 366. The one or more protrudent pillars 344 are wedged into one of the plurality of recesses 3684.

According to the above description, the first sliding block 32 and the second sliding block 36 have identical structures and features. The difference between the embodiment in FIG. 3 and FIG. 4 is only the location for disposing the sliding blocks with respect to the shaft.

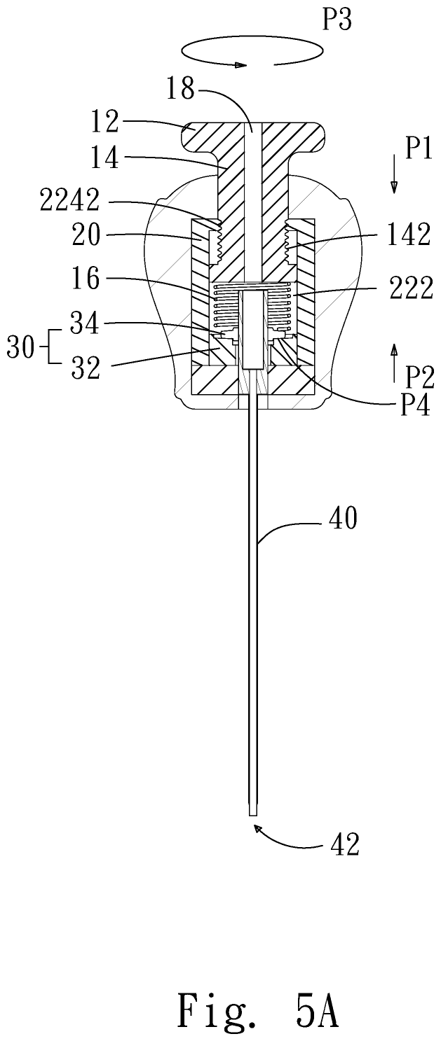

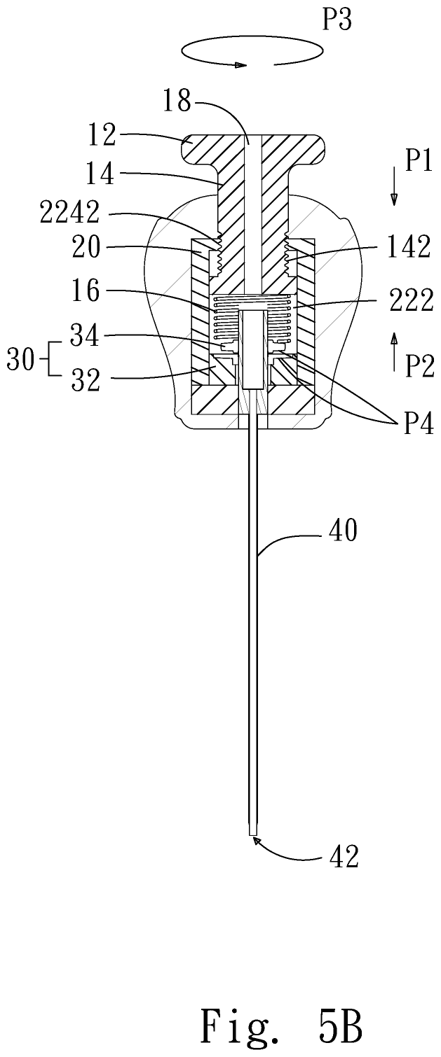

Please refer to FIG. 5A and FIG. 5B, which show schematic diagrams of the operation according to an embodiment of the present invention. As shown in the figures, the adjusting post 14 passes through the first opening 224. The outer thread 142 of the adjusting post 14 matches the inner thread 2242 in the first opening 224 such that the adjusting post 14 can turn downward into the accommodating space 222, making one end of the elastic member 16 against the adjusting post 14 and the other end against the torque damping device 30. Owing to the downward rotation, the adjusting post 14 exerts a downward pressure P1 on the elastic member 16. The torque damping device 30 and the elastic member 16 produce an upward resistive force. The downward pressure P1 and the upward pressure P2 make the elastic member 16 to produce elastic deformation. According to the elastic deformation of the elastic member 16, the screw torque of the hand tool can be adjusted. Then the holding part 20 can drive the driving shaft body 34 of the torque damping device 30 to screw an object.

Next, when a user exerts a torque P3 to the holding part 20 for screwing an object, since the one or more protrudent pillar 344 is wedged into one or more of the plurality of recessed 3284, the first sliding block 32 in the torque damping device 30 and the driving shaft body 34 will produce a torque P4 due to a reaction force. When the torque P4 is greater than the torque P3, the first sliding block 32 and the driving shaft body 34 have no relation rotation. Thereby, the user can screw using the hand tool normally. When torque P3 applied by the user is greater than the torque P4 produced by the reaction force, the first sliding block 32 slides off the driving shaft body 34 and relative rotation occurs. Thereby, given the predetermined torque value has been reached, the hand tool will not rotate.

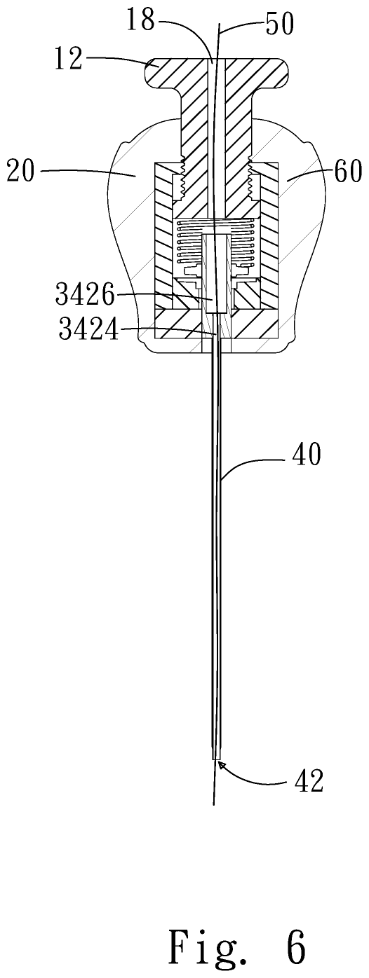

Next, please refer to FIG. 6, which shows an embodiment of the present invention. A through member 50 passes through the first hole 18, the counter bore 3426, the assembly hole 3424, and the second hole 42. The through member 50 can be medical rivets, sutures, or other medical sutures.

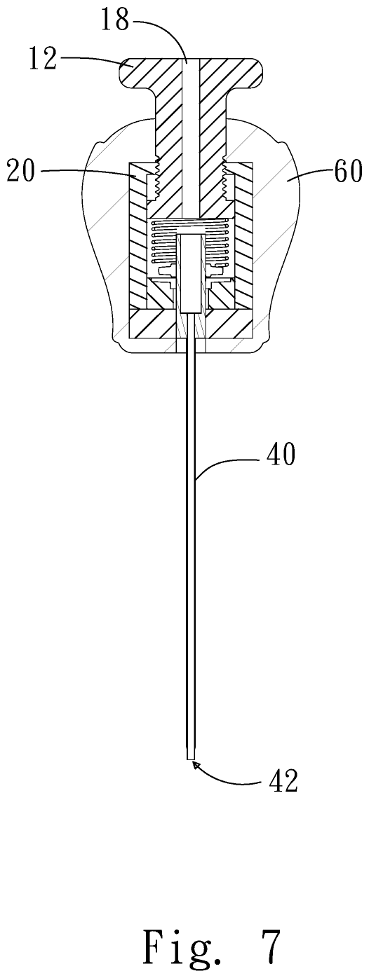

Next, please refer to FIG. 7, which shows an embodiment of the present invention. The holding part 20 further includes a hollow member 60 on the outer side. The hollow member 60 envelops the holding part 20 for facilitating force application on the medical torque hand tool.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

What is claimed is:

1. A medical torque hand tool, comprising:
a torque adjusting device, including an adjusting pole and an elastic member, and said adjusting pole including a first hole and against said elastic member;
a holding part, including a body and a blocking member, said body including an accommodating space, having a first opening on one end and a second opening on the other end, said blocking member including a bearing hole and communicating said second opening, said adjusting pole inserting said first opening, and said elastic member disposed in said accommodating space; and
a torque damping device, disposed in said accommodating space, said adjusting pole enabling said elastic member to compress and deform, and to against said torque damping device, and said torque damping device including a penetrating part communicating said first hole;
wherein when a user exerts an torque force on said holding part to screw an object, said torque damping device produces a torque by an inverse reaction force from said torque force; when said torque is greater than said torque force, said torque damping device is driven to carry a tool rotating along a direction of said torque force for screwing said object; and when said torque force is greater than said torque, said torque damping device idles and said tool stops to rotate.

2. The medical torque hand tool of claim 1, wherein said first opening further includes an inner thread on the inner side; said adjusting pole further includes an outer thread on the outer side; by matching said outer thread and said inner thread in said first opening, said adjusting pole can adjust the spring force exerted by said elastic member against said torque damping device.

3. The medical torque hand tool of claim 1, wherein said torque damping device includes:
a sliding block, having a block hole and an undulated structure surrounding said block hole, and said undulate structure including a plurality of slopes and a plurality of recessed arranged at intervals; and
a driving shaft body, including a spin shaft and one or more protrudent pillar, said penetrating part disposed at said spin shaft, said spin including an assembly hole at one end, said end of said spin shaft disposed in said block hole and said bearing hole, and said one or more protrudent pillar disposed on one side of said spin shaft;
wherein said one or more protrudent pillar is wedged in one of said plurality of recesses.

4. The medical torque hand tool of claim 3, wherein said sliding block and said elastic member clip said protrudent pillars.

5. The medical torque hand tool of claim 3, wherein said sliding block and said blocking member clip said protrudent pillars.

6. The medical torque hand tool of claim 4, further comprising a surgery tool inserting said assembly hole of said torque damping device.

7. The medical torque hand tool of claim 5, further comprising a surgery tool inserting said assembly hole of said torque damping device.

8. The medical torque hand tool of claim 6, wherein said spin shaft includes a counter bore opposing to said first hole at the other end; said assembly hole communicates said counter bore to form said penetrating part; said surgery tool includes a second hole; and a through member passes through said first hole, said penetrating part, and said second hole.

9. The medical torque hand tool of claim 7, wherein said spin shaft includes a counter bore opposing to said first hole at the other end; said assembly hole communicates said counter bore to form said penetrating part; said surgery tool includes a second hole; and a through member passes through said first hole, said penetrating part, and said second hole.

10. The medical torque hand tool of claim 1, further comprising a hollow member enveloping one side of said holding part.
